(19) United States
(12) Patent Application Publication
(10) Pub. No.: US 2019/0290194 A1
(43) Pub. Date: Sep. 26, 2019

(54) BIOMETRIC INFORMATION MEASURING APPARATUS AND NON-TRANSITORY COMPUTER READABLE MEDIUM

(71) Applicant: FUJI XEROX CO., LTD., Tokyo (JP)

(72) Inventors: Hideyuki UMEKAWA, Kanagawa (JP); Manabu AKAMATSU, Kanagawa (JP); Kazuhiro SAKAI, Kanagawa (JP); Hideaki OZAWA, Kanagawa (JP); Kazuyuki MATSUSHITA, Kanagawa (JP); Kohei YUKAWA, Kanagawa (JP)

(73) Assignee: FUJI XEROX CO., LTD., Tokyo (JP)

(21) Appl. No.: 16/294,199

(22) Filed: Mar. 6, 2019

(30) Foreign Application Priority Data
Mar. 23, 2018 (JP) 2018-057116
Mar. 23, 2018 (JP) 2018-057117

(51) Int. Cl. A61B 5/00; A61B 5/1455; A61B 5/087; A61B 5/0205; A61B 5/113

(52) U.S. Cl. CPC A61B 5/4818; A61B 5/742; A61B 5/14552; A61B 5/02116; A61B 5/0205; A61B 5/1135; A61B 5/087

(57) ABSTRACT

A biometric information measuring apparatus includes: a notification unit that notifies a subject holding breath to resume breathing when a specified time to hold breath is reached; and a measuring unit that measures an oxygen circulation time representing a time required for oxygen taken into a body of the subject by resumption of breathing by the subject to reach a predetermined site.

### CROSS-REFERENCE TO RELATED APPLICATIONS

This application is based on and claims priority under 35 USC 119 from Japanese Patent Application No. 2018-057116 filed Mar. 23, 2018 and Japanese Patent Application No. 2018-057117 filed Mar. 23, 2018.

### BACKGROUND

### (i) Technical Field

The present disclosure relates to a biometric information measuring apparatus and a non-transitory computer readable medium.

### (ii) Related Art

JP 2015-190413 A1 discloses a circulation time measuring apparatus including a signal acquiring unit that acquires an airflow signal indicating a temporal change of an airflow of breathing and an oxygen saturation signal indicating a temporal change of an oxygen saturation, and a circulation time calculating unit that measures an oxygen circulation time in blood based on a time difference between a first time in the airflow signal and a second time in the oxygen saturation signal indicating an increase of an oxygen saturation in accordance with a resumption of breathing at the first time.

In recent years, a measurement method for measuring biometric information has been developed. The measurement method is for measuring biometric information using an oxygen circulation time that indicates the time required for oxygen taken into a body to be transported to a predetermined site.

For example, the oxygen circulation time is estimated from the oxygen saturation. When the oxygen circulation time is measured using the oxygen saturation, it is preferable that the subject stops breathing before measurement of the oxygen circulation time to lower the oxygen saturation in advance so that an increase in the oxygen saturation due to the oxygen taken into the body can be detected.

However, if an actual breath holding period of the subject is too short, an amount of change in the oxygen saturation tends to be small; therefore, the measurement of the oxygen circulation time may become difficult. In addition, the amount of change in the oxygen saturation tends to increase as the breath holding period becomes long, and the oxygen circulation time is easily measured as the period is long. However, if the breath holding period is too long, a forcible holding of breathing affects, for example, a pulse wave to deviate a value thereof from the normal value; therefore, the oxygen circulation time may not be measured accurately.

A measurement method of the oxygen circulation time in the related art is performed by detecting a breathing state of the subject, for example, by a detecting unit such as an airflow sensor attached to a mouth or nose of a subject, using a period in which the subject becomes apneic during sleep.

However, since frequency and period of the apnea state during sleep vary depending on a subject, it is desirable to perform the measurement in a state in which the subject is awake in order to measure the oxygen circulation time in a more stable state.

In this case, since the detecting unit such as the airflow sensor is attached to the mouth or nose of the subject who is awake to detect a breathing state of the subject, a load on the subject is increased as compared with the case where the detecting unit that detects the breathing state is not attached to a face.

### SUMMARY

Aspects of non-limiting embodiments of the present disclosure relate to providing a biometric information measuring apparatus and a non-transitory computer readable medium that allow measurement of an oxygen circulation time with reduced variations in subject's breath holding period as compared to a case where the subject who is holding breath is not notified of when to resume breathing.

Aspects of non-limiting embodiments of the present disclosure also relate to providing a biometric information measuring apparatus and a non-transitory computer readable medium that allow measurement of an oxygen circulation time of a subject without detecting the breathing state of the subject.

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

According to one aspect of the present disclosure, there is provided a biometric information measuring apparatus including: a notification unit that notifies a subject holding breath to resume breathing when a specified time to hold breath is reached; and a measuring unit that measures an oxygen circulation time representing a time required for oxygen taken into a body of the subject by resumption of breathing by the subject to reach a predetermined site.

### BRIEF DESCRIPTION OF THE DRAWINGS

Exemplary embodiments of the present invention will be described in detail based on the following figures, wherein:

FIG. 1 is a schematic diagram showing an example of a measurement of an oxygen saturation in blood;

FIG. 2 is a graph showing an example of a change in an amount of light absorbed by a living body;

FIG. 3 is a diagram showing an example of the amount of light absorption in accordance with each wavelength of oxyhemoglobin and reduced hemoglobin;

FIG. 4A is a diagram showing a configuration example of a biometric information measuring apparatus according to a first exemplary embodiment;

FIG. 4B is a diagram showing another configuration example of the biometric information measuring apparatus according to the first exemplary embodiment;

FIG. 5 is a diagram showing an example of an arrangement of a light emitting element and a light receiving element;

FIG. 6 is a diagram showing another example of the arrangement of the light emitting element and the light receiving element;

FIG. 7 is a diagram showing an example of a breathing waveform;

FIG. 8 is a diagram showing an example of a change in an oxygen saturation in blood according to a stoppage of breathing and resumption of breathing;

FIG. 9 is a diagram showing a configuration example of a main part of an electrical system in the biometric information measuring apparatus;

FIG. 10 is a flowchart showing an example of a biometric information measurement processing according to the first exemplary embodiment and a second exemplary embodiment;

FIG. 11 is a diagram showing a configuration example of a biometric information measuring apparatus according to a third exemplary embodiment;

FIG. 12 is a flowchart showing an example of a biometric information measurement processing according to the third exemplary embodiment;

FIG. 13 is a flowchart showing another example of the biometric information measurement processing according to the third exemplary embodiment; and

FIG. 14 is a diagram showing still another configuration example of the biometric information measuring apparatus according to the third exemplary embodiment.

### DETAILED DESCRIPTION

Hereinafter, the present exemplary embodiment will be described with reference to the drawings. Components and processes having the same function are denoted by the same reference numerals throughout the drawings, and a repetitive description thereof is omitted.

### First Exemplary Embodiment

A biometric information measuring apparatus 10 is an apparatus for measuring biometric information particularly related to a circulatory system among information (biometric information) relating to a living body 8. The circulatory system collectively refers to a group of organs for transporting a body fluid such as blood while circulating the fluid in a body.

There are plural indicators in the biometric information on the circulatory system, and as one of the indicators indicating a state of a heart that pumps blood into the blood vessels, for example, the cardiac output (CO) indicating the amount of blood pumped out from the heart is mentioned.

Cardiac output has been used to test for various heart diseases or to confirm medication efficacy, for example, it is known that a decrease in cardiac output below a reference value is a suspicion of left heart failure, and an increase in cardiac output above a reference value is a suspicion of right heart failure.

As a method of measuring cardiac output, for example, a method is used in which a catheter with a balloon at a tip thereof is inserted into a pulmonary artery of a subject whose cardiac output is to be measured, an oxygen saturation in blood is measured while the balloon is inflated and deflated, and the cardiac output is calculated from the measured oxygen saturation. Here, the oxygen saturation in blood is an example of an index indicating the oxygen concentration of blood, and is an index indicating how much hemoglobin in blood is bound to oxygen, and indicates that as the oxygen saturation in blood decreases, symptoms such as anemia, for example, tend to occur.

However, in the method of measuring cardiac output using a catheter, since it is necessary to insert a catheter into a blood vessel of a subject, and to perform a surgical procedure; therefore, the invasiveness of the subject is increased as compared with other measurement methods.

Therefore, a method of measuring the cardiac output using the oxygen saturation obtained from a pulse wave of a subject has been studied so that the load on the subject is reduced compared with the method of measuring the cardiac output using a catheter. The pulse wave is an index indicating a pulsatile change of a blood vessel according to pumping of blood by the heart.

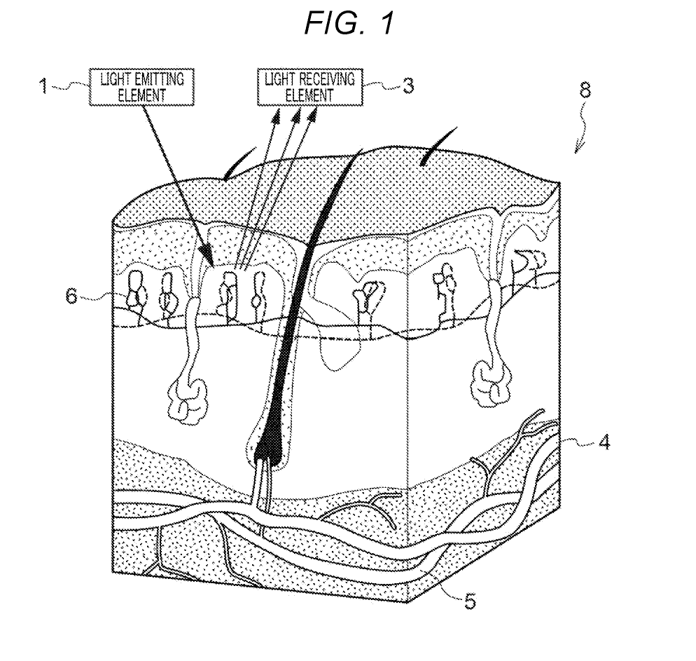

First, a method of measuring an oxygen saturation in blood among biometric information will be described with reference to FIG. 1.

As shown in FIG. 1, a light emitting element 1 emits light to a body (living body 8) of the subject and a light receiving element 3 receives the light to measure an oxygen saturation in blood by using the intensity of the light reflected or transmitted by an artery 4, a vein 5, a capillary 6, and the like that are stretched in the body of the subject, that is, an amount of received reflected light or transmitted light.

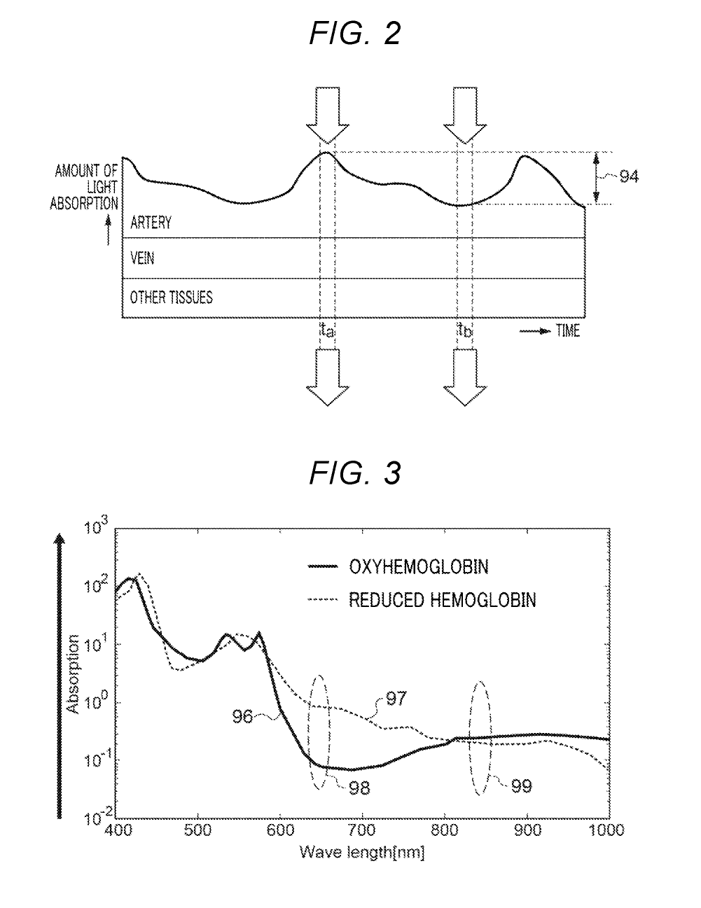

FIG. 2 is a conceptual diagram showing, for example, an amount of change of light absorbed by the living body 8. As shown in FIG. 2, the amount of light absorbed by the living body 8 tends to fluctuate over time.

Regarding the details of variations in the amount of light absorbed by the living body 8, it has been found that the amount of light absorption mainly by the artery 4 varies, and the amount of light absorption by other tissues including the vein 5 and the stationary tissue hardly varies as compared with the variation by the artery 4. This is because arterial blood pumped out from a heart moves in a blood vessel in accordance with the pulse wave, so that the artery 4 expands and contracts along the cross-sectional direction of the artery 4 over time, and the thickness of the artery 4 changes. In FIG. 2, a range indicated by an arrow 94 indicates the amount of change in the amount of light absorption corresponding to a change in the thickness of the artery 4.

In FIG. 2, when the amount of light received at time tₐ is represented by Iₐ and the amount of light received at time t_{b} is represented by I_{b}, a change amount ΔA of the light absorption by the change in the thickness of the artery 4 is represented by Equation (1).

ΔA=ln(I_{b}/Iₐ) (1)

On the other hand, FIG. 3 is a diagram showing an example of the amount of light absorption for each wavelength of hemoglobin (oxyhemoglobin) bound to oxygen flowing through the artery 4 and hemoglobin (reduced hemoglobin) not bound to oxygen. In FIG. 3, a graph 96 represents an amount of light absorption in oxyhemoglobin, and a graph 97 represents an amount of light absorption in reduced hemoglobin.

As shown in FIG. 3, it has been found that oxyhemoglobin tends to absorb light in an infrared (IR) region 99 having a wavelength about 850 nm compared to reduced hemoglobin, and reduced hemoglobin tends to absorb light in a red region 98 having a wavelength about 660 nm in particular compared to oxyhemoglobin.

Furthermore, it has been found that an oxygen saturation is proportional to the ratio of the change amount ΔA of the light absorption at different wavelengths.

Therefore, an oxygen saturation S is obtained based on Equation (2) by calculating the ratio of a change amount ΔA_{IR} and a change amount ΔA_{Red} using an infrared light (IR light) and a red light by which the difference in the amount of light absorption between the oxyhemoglobin and the reduced hemoglobin is likely to occur as compared with combinations of the other wavelengths. The change amount ΔA_{IR} is a change amount of the light absorption when the living body 8 is irradiated with the IR light and the change amount ΔA_{Red} is a change amount of the light absorption when the living body 8 is irradiated with the red light. In Equation (2), k is a proportionality constant.

S=k(ΔA_{Red}/ΔA_{IR}) (2)

That is, when calculating an oxygen saturation in blood, the living body 8 is irradiated with plural light having different wavelengths and emitted from the light emitting elements 1. Specifically, the light emitting element 1 for emitting IR light and the light emitting element 1 for emitting red light are used for the living body 8. In this case, light emitting periods of the light emitting element 1 for emitting IR light and the light emitting element 1 for emitting red light may overlap, but it is desirable that the light emitting periods do not overlap. Then, the light reflected or transmitted from each light emitting element 1 is received by the light receiving element 3, and an oxygen saturation is measured by calculating Equations (1) and (2) or a known equation obtained by modifying the equations from the amount of light received at each light-receiving point in time.

As a known equation obtained by modifying above Equation (1), for example, Equation (1) may be developed to express the change amount ΔA of the light absorption as Equation (3).

ΔA=ln I_{b}−ln Iₐ (3)

Equation (1) can be modified as Equation (4).

ΔA=ln(I_{b}/Iₐ)=ln(1+(I_{b}−Iₐ)/Iₐ) (4)

Normally, since (I_{b}−Iₐ)<<Iₐ, ln(L/I_{b}/Iₐ)/Iₐ are satisfied, instead of Equation (1), Equation (5) may be used to express the change amount ΔA of the light absorption.

ΔA≈(I_{b}−Iₐ)/Iₐ (5)

Hereinafter, when it is necessary to distinguish between the light emitting element 1 for emitting IR light and the light emitting element 1 for emitting red light, the light emitting element 1 for emitting IR light is referred to as a “light emitting element 1A” and the light emitting element 1 for emitting red light is referred to as a “light emitting element 1B”.

According to the method, since an oxygen saturation in blood is measured by bringing the light emitting element 1 and the light receiving element 3 close to the body surface of the subject, the burden on the subject is reduced compared to the case where an oxygen saturation in blood is measured by inserting a catheter into a blood vessel.

Then, using the measured oxygen saturation of the subject, the biometric information measuring apparatus 10 calculates a cardiac output by a method described later.

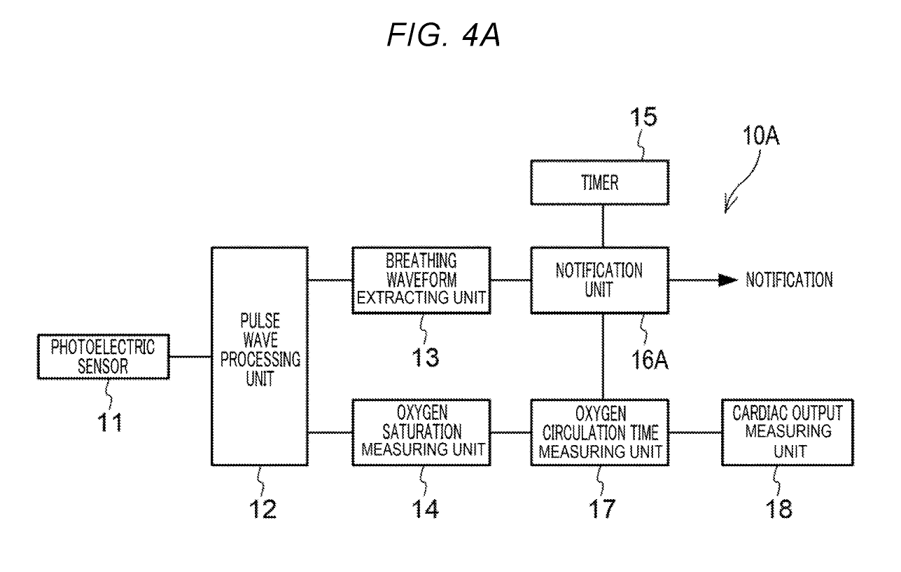

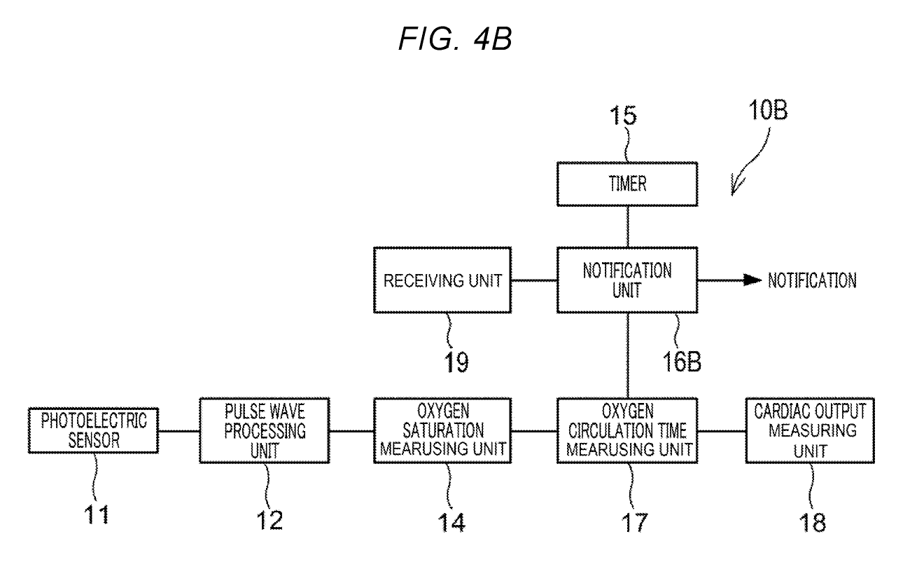

FIG. 4A is a diagram showing a configuration example of a biometric information measuring apparatus 10A. The biometric information measuring apparatus 10A shown in FIG. 4A includes a photoelectric sensor 11, a pulse wave processing unit 12, a breathing waveform extracting unit 13, an oxygen saturation measuring unit 14, a timer 15, a notification unit 16, an oxygen circulation time measuring unit 17, and a cardiac output measuring unit 18.

FIG. 4B is a diagram showing another configuration example of a biometric information measuring apparatus 10B. The biometric information measuring apparatus 10B shown in FIG. 4B includes the photoelectric sensor 11, the pulse wave processing unit 12, a receiving unit 19, the oxygen saturation measuring unit 14, the timer 15, the notification unit 16, the oxygen circulation time measuring unit 17, and the cardiac output measuring unit 18.

The photoelectric sensor 11 includes the light emitting element 1A for emitting IR light having a wavelength about 850 nm as a center wavelength, the light emitting element 1B for emitting red light having a wavelength about 660 nm as a center wavelength, and the light receiving element 3 for receiving IR light and red light.

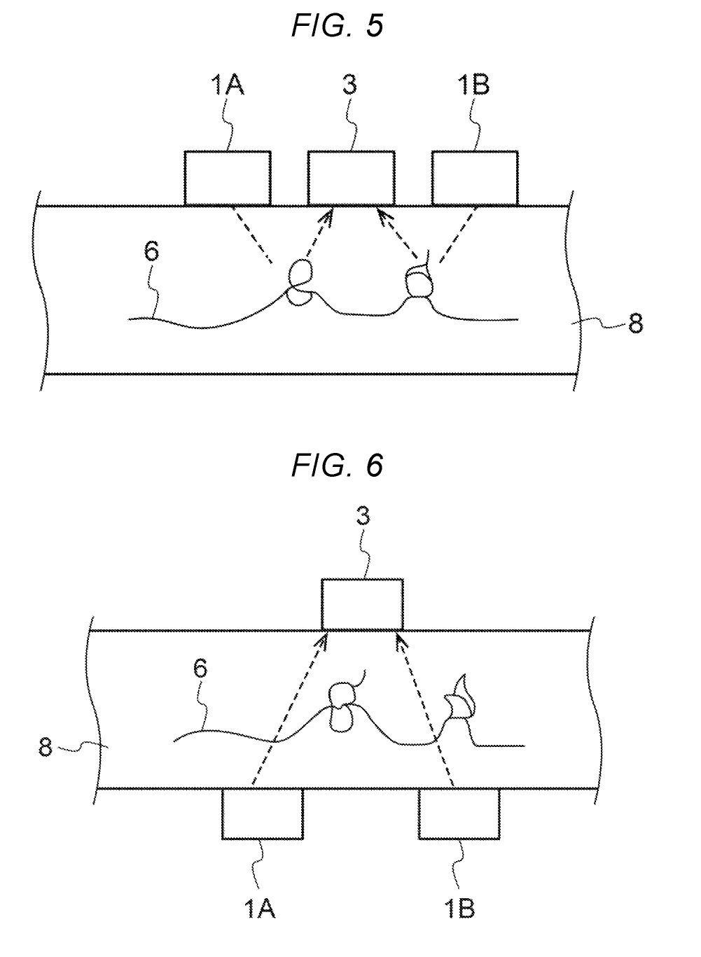

FIG. 5 shows an example of an arrangement of the light emitting element 1A, the light emitting element 1B, and the light receiving element 3 in the photoelectric sensor 11. As shown in FIG. 5, the light emitting element 1A, the light emitting element 1B, and the light receiving element 3 are arranged side by side along one surface of the living body 8. In this case, the light receiving element 3 receives the IR light and the red light reflected by the capillary 6 and the like of the living body 8.

However, the arrangement of the light emitting element 1A, the light emitting element 1B, and the light receiving element 3 is not limited to the arrangement example of FIG. 5. For example, as shown in FIG. 6, the light emitting element 1A, the light emitting element 1B, and the light receiving element 3 may be disposed at positions facing each other with the living body 8 interposed therebetween. In this case, the light receiving element 3 receives the IR light and the red light transmitted through the living body 8.

Here, as an example, the light emitting element 1A and the light emitting element 1B are described as surface emitting laser elements such as, vertical cavity surface emitting laser (VCSEL), but the present invention is not limited thereto, and may be edge emitting laser elements. The light emitting elements 1A and 1B may be a light emitting diode (LED).

The photoelectric sensor 11 is provided with a clip (not shown) for attaching the photoelectric sensor 11 to a body site of the subject, and the photoelectric sensor 11 is attached so as to be in contact with a body surface of the subject by a clip (not shown) so that IR light and red light do not leak from the photoelectric sensor 11 to the outside. In order to receive the IR light and the red light reflected or transmitted by the living body 8 of the subject as accurately as possible by the light receiving element 3, the photoelectric sensor 11 may be disposed in contact with the body surface of the subject, but the photoelectric sensor 11 may be attached at a position away from the body surface within a range in which the IR light and the red light reflected by the living body 8 of the subject or the IR light and the red light transmitted through the living body 8 of the subject are received by the light receiving element 3.

The photoelectric sensor 11 converts the respective received amounts of the IR light and the red light received by the light receiving element 3 into, for example, a voltage value, and notifies the pulse wave processing unit 12 of the voltage value.

Since predetermined amounts of light are emitted from the light emitting element 1A and the light emitting element 1B, the amounts of light absorption of IR light and red light in the living body 8 are obtained from the respective amounts of received IR light and red light received by the photoelectric sensor 11.

Therefore, the pulse wave processing unit 12 respectively generates a pulse wave signal indicating the pulse wave of the subject obtained from the IR light and a pulse wave signal indicating the pulse wave of the subject obtained from the red light, using the respective amounts of received IR light and red light received from the photoelectric sensor 11. The pulse wave processing unit 12 amplifies the voltage value so that the voltage value corresponding to the amounts of received IR light and red light is within a predetermined range suitable for generating the pulse wave signal. Then, the pulse wave processing unit 12 generates each pulse wave signal from which the noise component is removed by using a known filter or the like.

In the apparatus 10A of FIG. 4A, the pulse wave processing unit 12 notifies the breathing waveform extraction unit 13 and the oxygen saturation measuring unit 14 of each generated pulse wave signal. In the apparatus 10B of FIG. 4B, the pulse wave processing unit 12 notifies the oxygen saturation measuring unit 14 of each generated pulse wave signal.

Upon receiving the pulse wave signal from the pulse wave processing unit 12, the breathing waveform extracting unit 13 extracts a breathing waveform indicating the breathing state of the subject from the pulse wave signal.

Specifically, the breathing waveform extracting unit 13 detects a maximal value and a minimal value of any one of the pulse wave signal obtained from the IR light and the pulse wave signal obtained from the red light in a predetermined period (for example, 1 minute), and extracts a line (peak line) connecting each detected maximal value or a line (bottom line) connecting each detected minimal value as the breathing waveform of the subject.

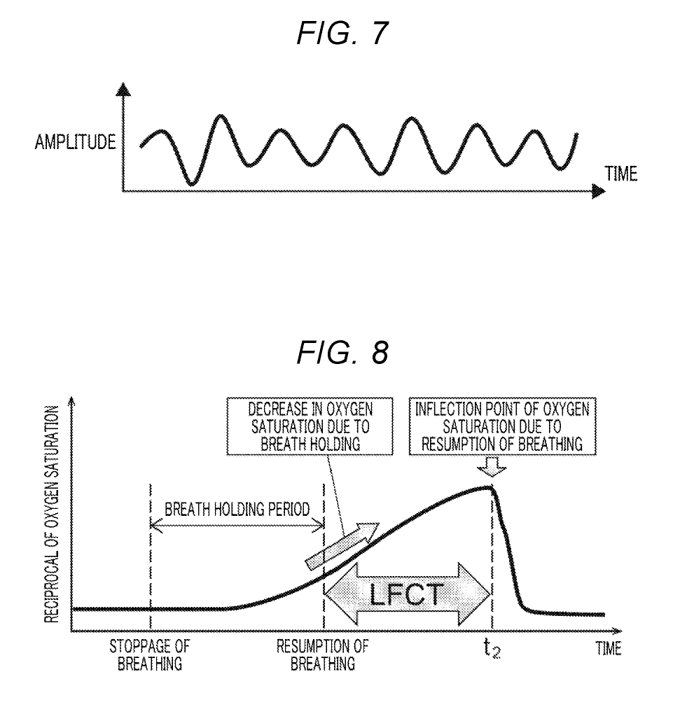

FIG. 7 is a diagram showing an example of a breathing waveform extracted from a pulse wave signal by the breathing waveform extracting unit 13.

The breathing waveform extracting unit 13 extracts the breathing waveform using the pulse wave signal obtained from the IR light. As shown in FIG. 3, since the IR light is more easily absorbed by the oxyhemoglobin than the red light, an amplitude of the pulse wave signal with respect to the change in the thickness of the artery 4 tends to be larger than an amplitude of the pulse wave signal obtained from the red light. Therefore, in the breathing waveform extracted from the pulse wave signal obtained from the IR light, the variation of the waveform becomes clearer than that of the breathing waveform extracted from the pulse wave signal obtained from the red light, and the highly accurate breathing waveform can be obtained.

The breathing waveform extracting unit 13 refers to the breathing waveform extracted from the pulse wave signal, and notifies the notification unit 16 of a breathing state of the subject, such as the stoppage of breathing and the resumption of breathing.

Since the pulse wave signal of the subject is obtained by the amount of received light detected by the photoelectric sensor 11 and the breathing state of the subject is detected from the pulse wave signal, the biometric information measuring apparatus 10 including the photoelectric sensor 11 can be said to be an example of a detecting unit that detects whether the subject is breathing.

When the pulse wave signal is received from the pulse wave processing unit 12, the oxygen saturation measuring unit 14 measures an oxygen saturation of the subject from the received pulse wave signal. Specifically, the oxygen saturation measuring unit 14 respectively calculates the change amount ΔA_{IR} of light absorption of IR light and the change amount ΔA_{Red} of light absorption of red light due to a change in the thickness of the artery 4 based on Equation (1), using the pulse wave signals. Then, the oxygen saturation measuring unit 14 measures the oxygen saturation of the subject based on Equation (2), for example, using the calculated change amount ΔA_{IR} and change amount ΔA_{Red}, and notifies the oxygen circulation time measuring unit 17 of the measured oxygen saturation.

Hereinafter, as an example, an example will be described in which the oxygen saturation measuring unit 14 measures the oxygen saturation of the subject, but the oxygen saturation measuring unit 14 may measure any value as long as the value indicates the temporal change of the oxygen saturation of the subject. For example, the oxygen saturation measuring unit 14 may measure a value correlated with the temporal change of the oxygen saturation, such as a reciprocal number of the oxygen saturation, or a ratio of the change amount ΔA_{Red} and the change amount ΔA_{IR}.

In the apparatus of FIG. 4B, the receiving unit 19 is an example of a receiving unit that receives the breathing state of the subject. Specifically, when the receiving unit 19 receives a notification to stop breathing via an input device operated by the subject or a measurer such as a medical professional who measures biometric information on the subject, the receiving unit 19 regards that the breathing of the subject is stopped. When the receiving unit 19 receives a notification to resume breathing from the input device, the receiving unit 19 regards that the breathing of the subject is resumed.

Then, the receiving unit 19 notifies the notification unit 16 of the breathing state of the subject, for example, the stoppage of breathing and the resumption of breathing.

Hereinafter, a subject and a measurer such as a medical professional who measures biometric information on the subject, may be referred to as a “user of the biometric information measuring apparatus 10”.

The timer 15 is an example of a measurement device for measuring time, and measures a cumulative time from a specified point in time.

When the notification unit 16 receives the notification of the stoppage of breathing of the subject from the breathing waveform extracting unit 13 or the receiving unit 19, the notification unit 16 starts the timer 15, and when the breath holding period reaches a specified time, the notification unit 16 issues a notification to resume breathing to the subject holding breath.

The graph of FIG. 8 shows an example of the change in the oxygen saturation in blood at a specific site of the subject, and a horizontal axis represents time and a vertical axis represents a reciprocal of the oxygen saturation.

When the subject stops breathing at time to, the oxygen saturation in blood of the subject begins to decrease. Even if the subject resumes breathing after the specified time (time t₁) predetermined as a period during which the subject stops breathing, it takes time for oxygen taken into blood by resuming breathing to reach a specific site from the lungs, so that the oxygen saturation in blood of the subject decreases even after the time t₁. Since the oxygen taken into blood by resuming breathing reaches a specific site from the lungs, the oxygen saturation in blood in the subject turns to increase. Assuming that the point where the oxygen saturation in blood changes from decreasing to increasing is referred to as an “inflection point” and the time at which the inflection point occurs is a time t₂, the oxygen circulation time is represented by the difference between the time t₁ and the time t₂.

That is, the oxygen circulation time represents a required time for oxygen to be transported from the lungs to a specific site, and is also referred to as “oxygen transportation time”.

The oxygen circulation time measured from the oxygen saturation tends to vary in measurement accuracy due to variations in breath holding period. Thus, the specified time to hold breath is set.

The specified time is the value obtained in advance by experiments using an actual biometric information measuring apparatus 10, computer simulations based on the design specifications of the biometric information measuring apparatus 10, or the like, which are performed to increase the accuracy of the measurement of the oxygen circulation time by the biometric information measuring apparatus 10.

Therefore, the notification unit 16 notifies the subject of the resumption of breathing as the breath holding period of the subject approaches the specified time, and also notifies the oxygen circulation time measuring unit 17 that the breathing of the subject is resumed.

When the notification unit 16 notifies the oxygen circulation time measuring unit 17 that the breathing of the subject is resumed, the oxygen circulation time measuring unit 17 stores the time when being notified of the resumption of breathing as the time t₁. Then, the oxygen circulation time measuring unit 17 monitors the oxygen saturation measured by the oxygen saturation measuring unit 14, and detects the inflection point of the oxygen saturation. The oxygen circulation time measuring unit 17 stores the time at which the inflection point of the oxygen saturation is detected as the time t₂, and measures the time represented by the difference between the time t₁ and the time t₂ as the oxygen circulation time. It should be noted that “detecting an inflection point” includes a case where a position slightly deviated from an inflection point is detected within a range in which the measurement of the oxygen circulation time is not substantially affected.

Then, the oxygen circulation time measuring unit 17 notifies the cardiac output measuring unit 18 of the measured oxygen circulation time. As described above, the oxygen circulation time measuring unit 17 is an example of a measuring unit that measures the oxygen circulation time.

The measurement site of the oxygen circulation time is determined by the attachment position of the photoelectric sensor 11 in the subject, but in the present exemplary embodiment, the photoelectric sensor 11 is mounted on the peripheral portion of the subject as an example. More specifically, the photoelectric sensor 11 is mounted on a fingertip, and the oxygen circulation time in a case where oxygen is transported from the lung to the fingertip is measured. This is because the oxygen circulation time becomes longer because the distance from the lung is longer than that of the other sites, and therefore, the oxygen circulation time with higher accuracy can be obtained as compared with a case where the photoelectric sensor 11 is attached to the other sites. The “peripheral site” refers to a site located on the peripheral side from a neck, a shoulder, and a hip joint of the subject's body.

Thus, the oxygen circulation times from the lung to the fingertip may be called particularly lung to finger circulation time (LFCT). In the present exemplary embodiment, the photoelectric sensor 11 is attached to the fingertip of the subject, and the LFCT is measured by the oxygen circulation time measuring unit 17, but the attachment site of the photoelectric sensor 11 is not limited to the fingertip. The photoelectric sensor 11 may be attached to any site of the subject as long as the obtained measurement error of the oxygen circulation time is within a predetermined range. The “fingertip” refers to a fingertip of a hand of the subject, but the photoelectric sensor 11 may be attached to a tip of a toe of a foot.

The cardiac output measuring unit 18 measures the cardiac output of the subject using the LFCT received from the oxygen circulation time measuring unit 17. The cardiac output is calculated by, for example, a previously obtained arithmetic expression representing the relation between the LFCT and the cardiac output.

Note that the cardiac output measuring unit 18 may measure information on the cardiac output in addition to the cardiac output. The “information on the cardiac output” is information that has a correlation with the cardiac output, and includes, for example, cardiac index and stroke volume.

The “cardiac index” is a value obtained by dividing the cardiac output of the subject by the body surface area of the subject in order to correct the difference in cardiac output due to a different physique of the subject. The “stroke volume” is a value indicating the amount of blood to be ejected to the artery 4 by one contraction of the heart, and is obtained by dividing the cardiac output by the heart rate of the subject for one minute.

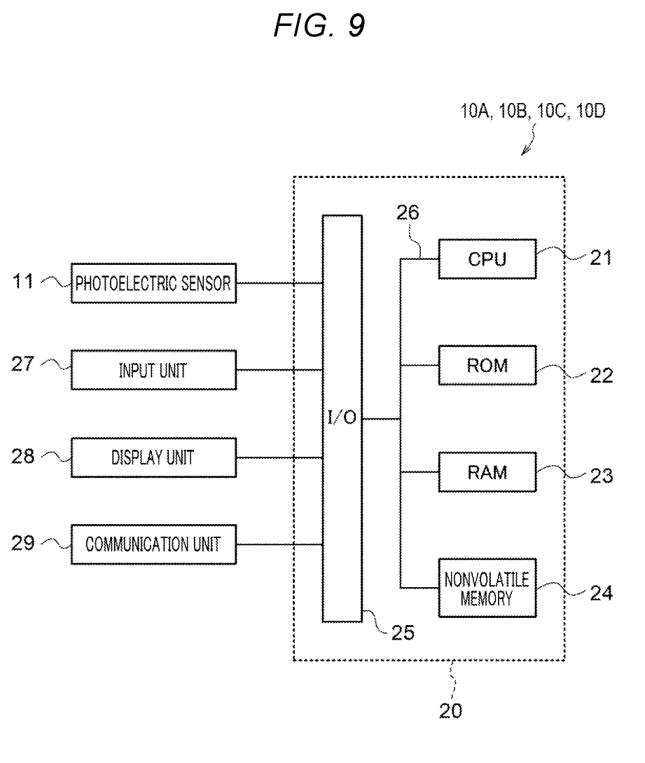

The above-described biometric information measuring apparatuses 10A and 10B are configured using, for example, a computer. FIG. 9 is a diagram showing an example of a configuration of a main part of an electrical system in the biometric information measuring apparatus 10 including a computer 20.

The computer 20 includes a central processing unit (CPU) 21, a read only memory (ROM) 22, a random access memory (RAM) 23, a nonvolatile memory 24, and an input/output interface (I/O) 25, which function as a notification unit, a measuring unit, and a detecting unit according to the present exemplary embodiment. The CPU 21, the ROM 22, the RAM 23, the nonvolatile memory 24, and the I/O 25 are connected to each other via a bus 26. The operation system used in the computer 20 is not limited.

The nonvolatile memory 24 is an example of a storage device that maintains stored information even when power supplied to the nonvolatile memory 24 is cut off, and for example, as the nonvolatile memory 24, a semiconductor memory is used, but a hard disk may also be used.

The I/O 25 is connected to, for example, the photoelectric sensor 11, an input unit 27, a display unit 28, and a communication unit 29.

The photoelectric sensor 11 is connected to the I/O 25 in a wired or wireless manner. Each of the biometric information measuring apparatus 10A or 10B and the photoelectric sensor 11 may be individually configured so as to be separated from each other, or may be configured to be accommodated in the same housing so that the biometric information measuring apparatus 10A or 10B and the photoelectric sensor 11 are integrated with each other.

The input unit 27 is, for example, an input device that receives an instruction from a user of the biometric information measuring apparatus 10A or 10B and notifies the CPU 21 of the instruction. The input unit 27 includes, for example, a button, a touch panel, a keyboard, a mouse, and the like.

As an example, the input unit 27 may include a button for notifying that breathing is stopped and a button for notifying that breathing is resumed. The user of the biometric information measuring apparatus 10A or 10B presses the button to notify the biometric information measuring apparatus 10 of the stoppage of breathing and the resumption of breathing. Hereinafter, a button for notifying that breathing is stopped is referred to as a “breathing stoppage button”, and a button for notifying that breathing is resumed is referred to as a “breathing resumption button”.

The CPU 21 may be notified of the stoppage of breathing and resumption of breathing by pressing a touch panel, pressing a keypad, or operating a mouse, for example, without necessarily being notified of the stoppage of breathing and resumption of breathing by the buttons.

Here, as the user, for example, a subject and an operator such as a medical professional who operates the biometric information measuring apparatus 10A or 10B are included.

The display unit 28 is, for example, a display device that visually displays information processed by the CPU 21 to the user of the biometric information measuring apparatus 10. As the display unit 28, for example, a display device such as a liquid crystal display, an organic electro luminescence (EL), or a projector is used.

The display unit 28 is not a unit necessary for the biometric information measuring apparatus 10A or 10B, and any type of unit may be connected to the I/O 25 as long as the unit notifies the user of information, such as a notification of resumption of breathing, issued from the biometric information measuring apparatus 10A or 10B.

For example, when information notified from the biometric information measuring apparatus 10A or 10B is notified to the user of the biometric information measuring apparatus 10A or 10B by voice, for example, a speaker unit may be connected the I/O 25 instead of the display unit 28. In addition, in the case of notifying the user of the information from the biometric information measuring apparatus 10A or 10B through a sense, for example, a vibration unit may be connected the I/O 25 instead of the display unit 28. Further, plural units such as the display unit 28 and the speaker unit may be used to notify the user of the information from the biometric information measuring apparatus 10A or 10B.

The communication unit 29 includes a communication protocol for connecting a communication line such as the Internet to the biometric information measuring apparatus 10A or 10B, and performs data communication between the biometric information measuring apparatus 10A or 10B and another external device connected to the communication line. The connection form to the communication line in the communication unit 29 may be wired or wireless. If the biometric information measuring apparatus 10A or 10B does not need to perform data communication with another external device connected to the communication line, the communication unit 29 does not need to be connected to the I/O 25.

The unit connected to the I/O 25 is not limited to the examples described above, and other units such as a printing unit may be connected to the I/O 25.

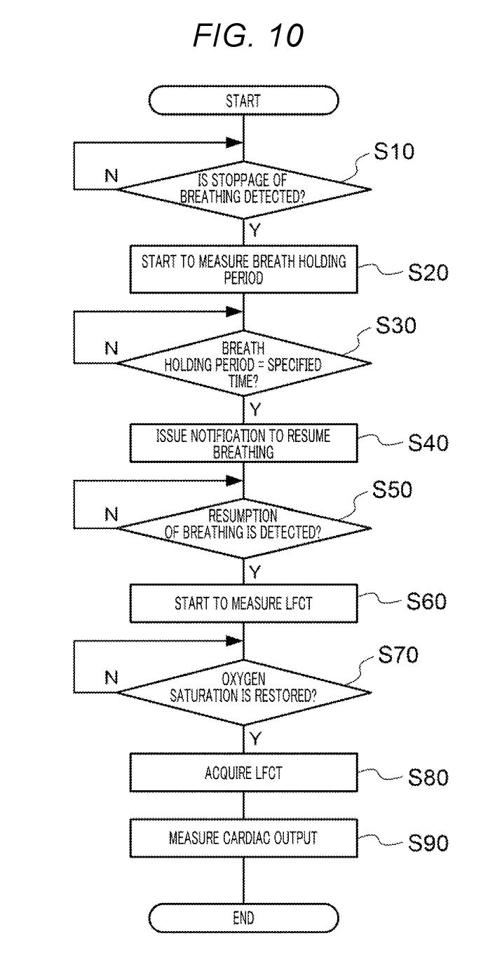

Next, the operation of the biometric information measuring apparatus 10 will be described with reference to FIG. 10.

FIG. 10 is a flow chart showing an example of a biometric information measurement processing executed by the CPU 21 when an instruction to measure the cardiac output is received from the user of the biometric information measuring apparatus 10A or 10B via the input unit 27 while the photoelectric sensor 11 is attached to the fingertip of the subject. When the biometric information measuring apparatus 10 receives an indication of the cardiac output, the oxygen saturation of the subject is continuously measured at least until the measurement of cardiac output is completed.

The biometric information measurement programs specifying the biometric information measurement processing are stored in advance in, for example, the ROM 22 of the biometric information measuring apparatus 10. The CPU 21 of the biometric information measuring apparatus 10 reads the biometric information measurement program stored in the ROM 22, and executes the biometric information measurement processing.

First, in step S10, the CPU 21 of the apparatus 10A refers to the breathing waveform obtained from the pulse wave signal detected by the photoelectric sensor 11, and determines whether or not the subject stopped breathing according to the measurement of the cardiac output.

When it is determined that the subject does not stop breathing, the processing of step S10 is repeatedly executed to monitor the breathing waveform of the subject. On the other hand, if it is determined that the subject stops breathing, the process proceeds to step S20.

For example, the CPU 21 detects whether the subject is breathing by a change in the amplitudes of the pulse waves, but the method of detecting whether the subject is breathing is not limited thereto.

For example, an airflow sensor for detecting the flow of airflow or an air quantity sensor for detecting a quantity of air may be attached to the mouth or nose of the subject, and whether the subject is breathing may be directly detected. Further, when the subject breathes, air warmed in the body is discharged from the mouth or nose, and therefore, whether the subject is breathing is directly detected by attaching a temperature sensor to the mouth or nose of the subject. Further, since a position of the chest of the subject varies due to breathing, whether the subject is breathing is detected by analyzing an image capturing the chest of the subject by a camera or attaching a displacement meter to the chest of the subject.

As described above, the airflow sensor, the air quantity sensor, the temperature sensor, the camera, and the displacement meter used for detecting whether the subject is breathing are also examples of the detecting unit like the photoelectric sensor 11.

On the other hand, in step S10, the CPU 21 of the apparatus 10B determines whether or not the subject stops breathing based on whether or not the breathing stoppage button included in the input unit 27 is pressed.

When the breathing stoppage button is not pressed, that is, when the subject does not stop breathing, the processing of step S10 is repeatedly executed to monitor the pressing state of the breathing stoppage button.

On the other hand, when the breathing stoppage button is pressed, that is, when it is determined that the subject stops breathing, the processing proceeds to step S20.

In step S20, the CPU 21 starts the timer 15 to measure the breath holding period. There is no limitation on the method for measuring the breath holding period, and the CPU 21 may measure the breath holding period using, for example, a timer function built in the CPU 21, or may measure the breath holding period using an external timer unit provided separately from the biometric information measuring apparatus 10 and connected to the I/O 25.

In step S30, the CPU 21 refers to the timer value of the timer 15 to determine whether or not the breath holding period reaches the specified time. When the breath holding period does not reach the specified time, the processing of step S30 is repeatedly executed to monitor the breath holding period of the subject. On the other hand, when the breath holding period reaches the specified time, the processing proceeds to step S40.

In this case, in order to make the subject to resume breathing, the CPU 21 notifies the subject of a resumption of breathing in step S40. More specifically, when the display unit 28 is connected to the biometric information measuring apparatus 10, the CPU 21 displays, for example, a message or a drawing prompting the resumption of breathing on the display unit 28. When the speaker unit is connected to the biometric information measuring apparatus 10, the CPU 21 outputs, for example, a sound prompting the resumption of breathing from the speaker unit.

In step S50, the CPU 21 of the apparatus 10A refers to the breathing waveforms obtained from the pulse wave signals detected by the photoelectric sensor 11, and determines whether or not the subject actually resumes breathing.

When it is determined that the subject does not resume breathing, the processing of step S50 is repeatedly executed to monitor the breathing waveform of the subject. On the other hand, if it is determined that the subject resumes breathing, the processing proceeds to step S60.

When the airflow sensor, the air quantity sensor, the temperature sensor, the camera, or the displacement meter is used for detecting whether the subject is breathing, the CPU 21 may detect resumption of breathing of the subject from changes in the sensor values.

In step S50, the CPU 21 of the apparatus 10B determines whether or not the subject resumes breathing based on whether or not the breathing resumption button included in the input unit 27 is pressed.

When the breathing resumption button is not pressed, that is, when the subject does not resume breathing, the processing of step S50 is repeatedly executed to monitor the pressing state of the breathing resumption button.

On the other hand, when the breathing resumption button is pressed, that is, when it is determined that the subject resumes breathing, the processing proceeds to step S60.

In step S40, the subject is notified to resume breathing at the timing when the breath holding period reaches the specified time. However, if the notification to resume breathing is suddenly issued, a delay may occur between the reception of the notification to resume breathing by the subject and the actual resumption of breathing. Therefore, the CPU 21 may sequentially display the remaining time until the specified time is reached on the display unit 28 to notify the subject of how long to hold breath during the breath holding period, and may notify the subject of the ending time of the breath holding period in advance.

Along with the resumption of breathing of the subject, in step S60, the CPU 21 acquires the time t₁ at which the resumption of breathing of the subject is detected, and stores the acquired time t₁ in the RAM 23, which is an example of storage device.

Here, there is no limitation on the method for acquiring the time information, and the CPU 21 may acquire the time information using, for example, a clock function built in the CPU 21, or may acquire the time information from an external clock unit connected to the I/O 25, which is prepared separately from the biometric information measuring apparatus 10A or 10B. In addition, the CPU 21 may acquire time information from a time server, which is an example of an external device connected to the communication line via the communication unit 29. Furthermore, a timer 15 may be used.

The time point at which the resumption of breathing is detected does not mean only one point of the moment at which the resumption of breathing is detected, but also means a predetermined period after the time at which the resumption of breathing is detected, a period including a delay time at which signal processing or the like related to the detection is performed, or the like. Further, since the time at which the subject resumes breathing is measured by pressing the breathing resumption button, the pressing of the breathing resumption button by the user of the biometric information measuring apparatus 10 is an example of the measurement instruction.

In step S70, the CPU 21 refers to the oxygen saturation obtained from the pulse wave signals detected by the photoelectric sensor 11, and determines whether or not the inflection point of the oxygen saturation is detected, in other words, whether or not the oxygen saturation is changed from decreasing to recovering.

If the oxygen saturation continues to decrease and the inflection point is not detected, the processing of step S70 is repeatedly executed to monitor the change in the oxygen saturation. On the other hand, when the inflection point of the oxygen saturation is detected, the processing proceeds to step S80.

In step S80, the CPU 21 acquires the time t₂ at which the inflection point of the oxygen saturation is detected, and stores the acquired time t₂ in the RAM 23. Since the time t₂ is the time at which the inflection point of the oxygen saturation occurs, the time t₂ is also referred to as “the time at which the inflection point occurs”. The time point at which the inflection point of the oxygen saturation is detected means not only one point at the moment at which the inflection point of the oxygen saturation is detected, but also a predetermined period after the time at which the inflection point of the oxygen saturation is detected, a period including a delay time at which signal processing or the like related to the detection is performed or the like.

The CPU 21 acquires the difference between the time t₂ and the time t₁ stored in the RAM 23 in step S60 as a LFCT.

In step S90, the CPU 21 uses the LFCT acquired in step S80 to measure the cardiac output based on, for example, Equation (6). In addition, the CPU 21 may calculate information on the cardiac output using the measured cardiac output.

Thus, the biometric information measurement processing shown in FIG. 10 is completed.

The breath holding period during which the LFCT is accurately measured varies according to, for example, age, sex, and physical condition of the subject. Therefore, based on the information on the subject set in the biometric information measuring apparatus 10 by the user of the biometric information measuring apparatus 10 via the input unit 27, the CPU 21 may adjust the specified time to hold breath for each subject. Alternatively, the user of the biometric information measuring apparatus 10 may adjust the specified time.

The specified time may be set per a second, for example, and a time selected from plural times prepared in advance, such as 15 seconds, 20 seconds, and 25 seconds may be set as the specified time. The setting unit of the specified time is not limited, and may be, for example, milliseconds or 5 seconds.

The specified time set for each subject is stored in, for example, the nonvolatile memory 24, and when information for identifying the subject, such as the name of the subject or the identification number of the patient, is inputted to the biometric information measuring apparatus 10 prior to the instruction to measure the cardiac output, the CPU 21 makes the specified time associated with the subject be used for the determination of step S30 of FIG. 10.

As shown in step S50 of FIG. 10, the CPU 21 starts the measurement of the LFCT after waiting for the subject to actually resume breathing, but if the period from when the biometric information measuring apparatus 10 notifies the subject of the resumption of breathing to when the subject resumes breathing is delayed, the breath holding period becomes longer than the specified time, and the measurement accuracy of the LFCT may be lower than when the breath holding period is adjusted to the specified time.

Therefore, the CPU 21 may measure the LFCT if the period from when the notification to resume breathing is issued to when the subject resumes breathing is within a predetermined allowable delay period. In other words, if the period from when the notification to resume breathing is issued to when the subject resumes breathing exceeds the allowable delay period, the CPU 21 may stop executing the processing of step S60 and subsequent steps in FIG. 10, and end the biometric information measurement processing shown in FIG. 10 without measuring the LFCT.

According to the biometric information measuring apparatus 10A of the first exemplary embodiment, the breathing state and the oxygen saturation in blood of the subject are measured using the pulse wave obtained from the sensor value of the photoelectric sensor 11, and the cardiac output is measured from the breathing state and the oxygen saturation in blood of the subject.

In addition, the biometric information measuring apparatus 10A notifies the subject of the resumption of breathing in accordance with the specified time.

According to the biometric information measuring apparatus 10B of the first exemplary embodiment, the breathing state and the oxygen saturation in blood of the subject are measured using the pulse wave obtained from the sensor value of the photoelectric sensor 11 in accordance with the instruction from the user, and the cardiac output is measured from the breathing state and the oxygen saturation in blood of the subject.

Various modifications are applied to the biometric information measurement processing shown in FIG. 10.

As shown in step S50 of FIG. 10, the CPU 21 waits for the user of the biometric information measuring apparatus 10B to press the breathing resumption button, and then starts the measurement of the LFCT. However, if the period from when the biometric information measuring apparatus 10B notifies the subject of the resumption of breathing to when the breathing resumption button is pressed is delayed, the breath holding period becomes longer than the specified time, and the measurement accuracy of the LFCT may be lower than when the breath holding period is adjusted to the specified time.

Therefore, the CPU 21 may measure the LFCT if the period from when the notification to resume breathing is issued to when the subject resumes breathing is within a predetermined allowable delay period. In other words, if the period from when the notification to resume breathing is issued to when the breathing resumption button is pressed exceeds the allowable delay period, the CPU 21 may stop executing the processing of step S60 and subsequent steps in FIG. 10, and end the biometric information measurement processing shown in FIG. 10 without measuring the LFCT.

Further, in the above-described biometric information measurement processing, the LFCT is measured from the difference between the time t₁ at which the resumption of breathing of the subject is detected and the time t₂ at which the inflection point of the oxygen saturation occurs, but the CPU 21 may restart the timer 15 instead of storing the time t₁ in step S60 in FIG. 10, and may stop the timer 15 instead of storing the time t₂ at which the inflection point occurs in step S80. In this case, the cumulative time from when the user of the biometric information measuring apparatus 10B presses the breathing resumption button to when the inflection point of the oxygen saturation occurs, which is measured by the timer 15, may be acquired as the LFCT.

On the contrary, in the above-described biometric information measurement processing, the breath holding period of the subject is measured using the timer 15, but the breath holding period of the subject may be measured using the absolute time. For example, instead of starting the timer 15 in step S20 in FIG. 10, the CPU 21 may store the time to at which the breathing stoppage button is pressed in the RAM 23, and may determine whether or not the difference between the time at which determination in step S30 is performed and the time to stored in the RAM 23 reaches the specified time in step S30.

In addition, in some cases, the subject resumes breathing a little after pressing the breathing resumption button, without resuming breathing in accordance with the pressing of the breathing resumption button. Therefore, the biometric information measuring apparatus 10 may measure the LFCT by considering the delay time from when the breathing resumption button is pressed to when the subject actually resumes breathing.

Specifically, the CPU 21 may acquire a time obtained by subtracting a delay time, which is a predetermined time, from the LFCT acquired in step S80 in FIG. 10 as a final LFCT. The delay time represents a time from when the breathing resumption button is pressed to when the subject actually resumes breathing, and is a value obtained in advance by an experiment using an actual biometric information measuring apparatus 10 for plural subjects.

In the biometric information measurement processing shown in FIG. 10, the oxygen saturation of the subject at each time is monitored in real time, and the cardiac output of the subject is measured during the measurement of the oxygen saturation, that is, so-called real time processing is performed. However, the biometric information measuring apparatus 10 does not necessarily need to perform real-time processing for measuring the cardiac output of the subject during the measurement of the oxygen saturation.

For example, the CPU 21 stores, in the RAM 23, the oxygen saturation and time information in association with each other at each time in the predetermined period after at least the breathing resumption button is pressed, and temporarily ends the biometric information measurement processing shown in FIG. 10 without executing steps S70 to S90.

When the CPU 21 receives an instruction to measure the cardiac output from the user of the biometric information measuring apparatus 10, the CPU 21 reads the oxygen saturation and the time information from the RAM 23 at each time after the breathing resumption button is pressed. Thereafter, the CPU 21 executes the processing including steps S70 to S90 in FIG. 9, detects the inflection point of the oxygen saturation from the read change of the oxygen saturation, acquires the LFCT from the difference between the time at which the breathing resumption button is pressed, that is, the time t₁ at which the resumption of breathing of the subject is detected and the time t₂ at which the inflection point of the oxygen saturation occurs, and measures the cardiac output using the acquired LFCT.

Here, the “predetermined period” is a period set longer than the longest LFCT that can be medically assumed.

The storage destination of the oxygen saturation and the time information may be the nonvolatile memory 24. Further, the CPU 21 may store the oxygen saturation and the time information in association with each other in a storage device, which is an example of an external device connected to the communication line, via the communication unit 29.

In the above example, the oxygen saturation and the time information are stored in the RAM 23 in association with each other, but since the oxygen saturation is obtained from the pulse wave signal, the pulse wave signal and the time information may be stored in the RAM 23 in association with each other. In this case, the LFCT and the cardiac output are measured on the basis of the oxygen saturation changes obtained from the pulse wave signals.

The biometric information measuring apparatus 10B according to the first exemplary embodiment receives the notification of the state of breathing from the user via the input unit 27. Then, when the instruction to resume breathing is issued, the biometric information measuring apparatus 10B measures the time from the reception of the instruction to the occurrence of the inflection point of the oxygen saturation, and measures the oxygen circulation time and the cardiac output. Therefore, in the biometric information measuring apparatus 10B, the oxygen circulation time and the cardiac output of the subject are measured without using a detecting unit such as an airflow sensor for detecting the breathing state of the subject.

### Second Exemplary Embodiment

The biometric information measuring apparatus 10A according to the first exemplary embodiment measures the breathing state of the subject using the photoelectric sensor 11, starts the measurement of the breath holding period when the stoppage of breathing is detected, and starts the measurement of the oxygen circulation time when the resumption of breathing is detected.

However, there is a case where the breathing state of the subject is obtained without using a detecting unit for detecting whether the subject is breathing such as the photoelectric sensor 11.

The second exemplary embodiment further relates to a biometric information measuring apparatus 10B shown in FIG. 4B, in which the breathing state of the subject is measured without using the detecting unit such as a photoelectric sensor 11 that detects whether the subject is breathing, and the notification to resume breathing is issued at the end of the specified time.

The biometric information measuring apparatus 10B shown in FIG. 4B includes the receiving unit 19 instead of the breathing waveform extraction unit 13 of the biometric information measuring apparatus 10A shown in FIG. 4A.

In the second exemplary embodiment, the input unit 27 includes an input member operated by the subject to notify the biometric information measuring apparatus 10A of the stoppage of breathing and the resumption of breathing. As an example, the subject presses a button to notify the biometric information measuring apparatus 10B of the stoppage of breathing and the resumption of breathing.

The input unit 27 notifies the receiving unit 19 of the operation state of the button operated by the subject.

The receiving unit 19 is an example of a receiving unit that receives the breathing state of the subject according to the operation state of the button notified from the input unit 27. More specifically, when the button associated with the stoppage of breathing is pressed, the receiving unit 19 determines that the breathing of the subject is stopped. When the button associated with the resumption of breathing is pressed, the receiving unit 19 determines that the breathing of the subject is resumed.

Then, the receiving unit 19 notifies the notification unit 16 of the breathing state of the subject, for example, the stoppage of breathing and the resumption of breathing.

The notification unit 16 starts the timer 15 when being notified of the stoppage of breathing of the subject by the receiving unit 19, and notifies the subject who is holding the breath of the resumption of breathing when the breath holding period reaches the specified time. When the receiving unit 19 notices the notification unit 16 of the resumption of breathing of the subject, the notification unit 16 notifies the oxygen circulation time measuring unit 17 of the resumption of breathing of the subject.

The biometric information measuring apparatus 10B is configured using, for example, a computer, similarly to the biometric information measuring apparatus 10A. In this case, the configuration example of the main part of the electrical system in the biometric information measuring apparatus 10B is the same as the configuration example of the main part of the electrical system in the biometric information measuring apparatus 10A shown in FIG. 9.

In the biometric information measuring apparatus 10B, the biometric information measurement processing may be performed according to the flowchart shown in FIG. 10.

In step S10 of FIG. 10, the biometric information measuring apparatus 10A determines whether or not the subject stops breathing with reference to the breathing waveform obtained from the pulse wave signal detected by the photoelectric sensor 11. On the other hand, the biometric information measuring apparatus 10B determines whether or not the subject stops breathing with reference to the pressed state of the button associated with the stoppage of breathing. In step S50 of FIG. 10, the biometric information measuring apparatus 10A determines whether or not the subject resumes breathing with reference to the breathing waveform obtained from the pulse wave signal detected by the photoelectric sensor 11. On the other hand, the biometric information measuring apparatus 10B determines whether or not the subject resumes breathing with reference to the pressed state of the button associated with the resumption of breathing.

The button for notifying the biometric information measuring apparatus 10B of the breathing state of the subject may be operated by the measurer who measures biometric information such as a medical professional who confirms the breathing state of the subject instead of the subject.

It should be noted that the contents of various modifications relating to the biometric information measurement processing described in the first exemplary embodiment are also applied to the biometric information measuring apparatus 10B.

As described above, according to the biometric information measuring apparatus 10B of the second exemplary embodiment, based on the report of the breathing state of the subject by the subject or the measurer who measures the biometric information, the subject is notified of the resumption of breathing in accordance with the specified time, and the cardiac output of the subject is measured.

### Third Exemplary Embodiment

In the biometric information measuring apparatus 10A according to the first exemplary embodiment and the biometric information measuring apparatus 10B according to the second exemplary embodiment, the measurement of the breath holding period and the measurement of the LFCT are started in accordance with the breathing state of the subject.

In the third exemplary embodiment, since the subject adjusts the breathing state to the instruction issued by the biometric information measuring apparatus, the measurement of the breath holding period and the measurement of the LFCT are started without confirming the breathing state of the subject to measure the cardiac output of the subject.

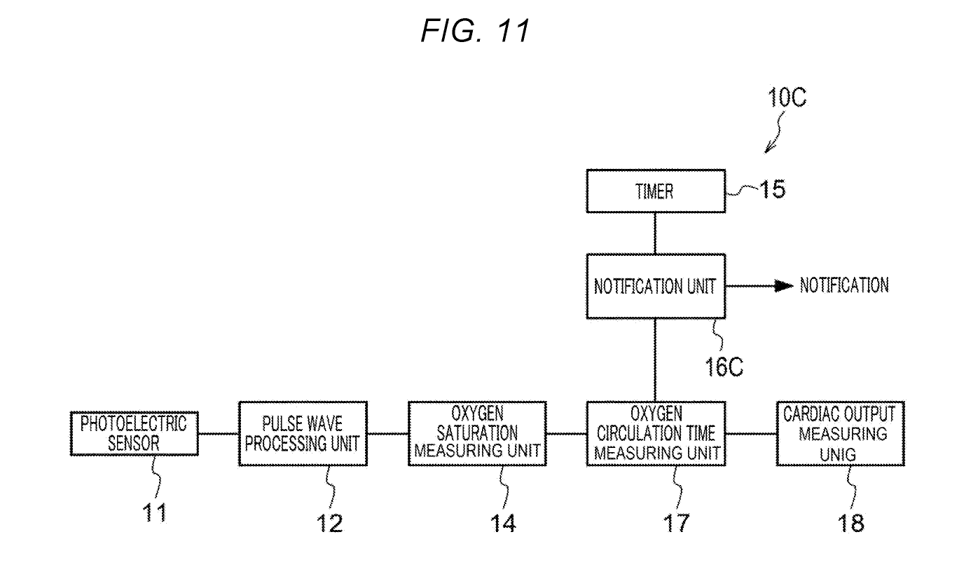

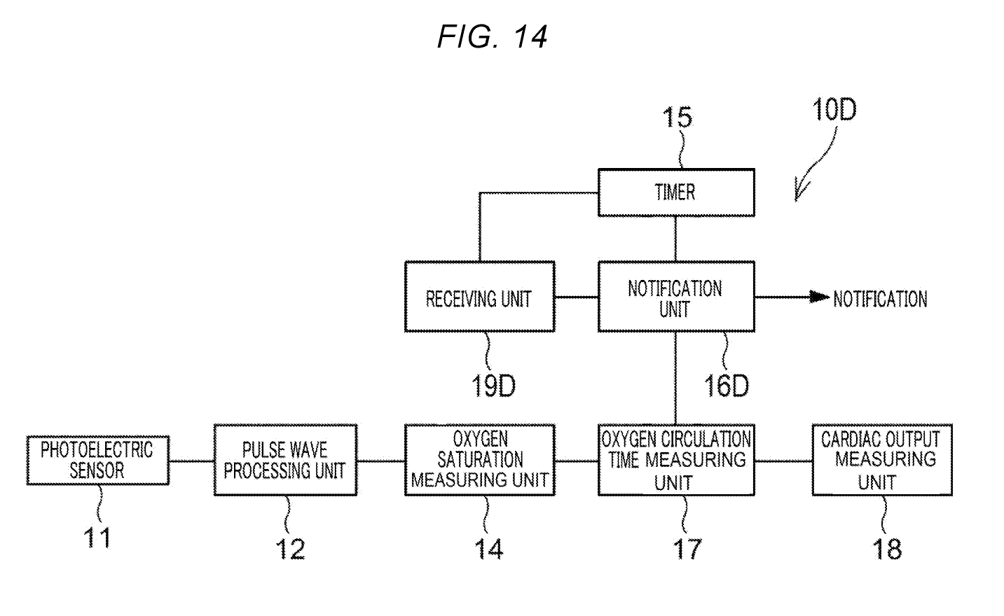

The third exemplary embodiment uses a biometric information measuring apparatus 10C or 10D shown in FIG. 11 or 14. The biometric information measuring apparatus 10C shown in FIG. 11 is different from the biometric information measuring apparatus 10A shown in FIG. 4A in that the biometric information measuring apparatus 10C includes a notification unit 16C instead of the breathing waveform extracting unit 13. The biometric information measuring apparatus 10D illustrated in FIG. 14 includes a different receiving unit 19D and a notification unit 16D, and the timer 15 and the receiving unit 19D are connected to each other, which is a point different from the biometric information measuring apparatus 10B illustrated in FIG. 4B.

Upon receiving the cardiac output measurement instruction, the receiving unit 19D notifies the notification unit 16D of the start of the cardiac output measurement. The receiving unit 19D receives the timer value measured by the timer 15 and notifies the notification unit 16D of the timer value, and when the timer value reaches the specified time, notifies the notification unit 16D of the start of measurement of the oxygen circulation time. That is, the timer value received by the receiving unit 19A from the timer 15 functions as an instruction to start measurement of the oxygen circulation time.

When the notification unit 16C receives the cardiac output measurement instruction, the notification unit 16C issue the notification to stop breathing in accordance with a predetermined rule and starts the timer 15, and when the timer value of the timer 15 reaches the specified time, the notification unit 16C notifies the subject of the resumption of breathing and also notifies the oxygen circulation time measuring unit 17 that the subject resumes breathing.

When the notification unit 16D receives the cardiac output measurement instruction, the notification unit 16D issues the notification to stop breathing in accordance with a predetermined rule and starts the timer 15, and when the timer value reaches the specified time and the start of measurement of the oxygen circulation time is notified from the receiving unit 19A, the notification unit 16D notifies the subject of the resumption of breathing and also notifies the oxygen circulation time measuring unit 17 that the subject resumes breathing.

Here, the “a predetermined rule” is a rule that determines the timing for issuing the notification to stop breathing, for example, the stoppage of breathing is notified n seconds (n is an integer equal to or greater than 0) later after receiving the instruction to measure the cardiac output. The value of n seconds that determines the timing for issuing the notification to stop breathing is called a “standby time” and is a value changed by the user of the biometric information measuring apparatus 10B.

The oxygen circulation time measuring unit 17 starts measuring the LFCT when the oxygen circulation time measuring unit 17 receives that the breathing of the subject is resumed from a notification unit 16B, and the cardiac output of the subject is obtained by the cardiac output measuring unit 18.

The biometric information measuring apparatus 10C or 10D is configured using, for example, a computer, similarly to the biometric information measuring apparatus 10A. The biometric information measuring apparatus 10C or 10D may have the components shown in FIG. 9 in the electrical system.

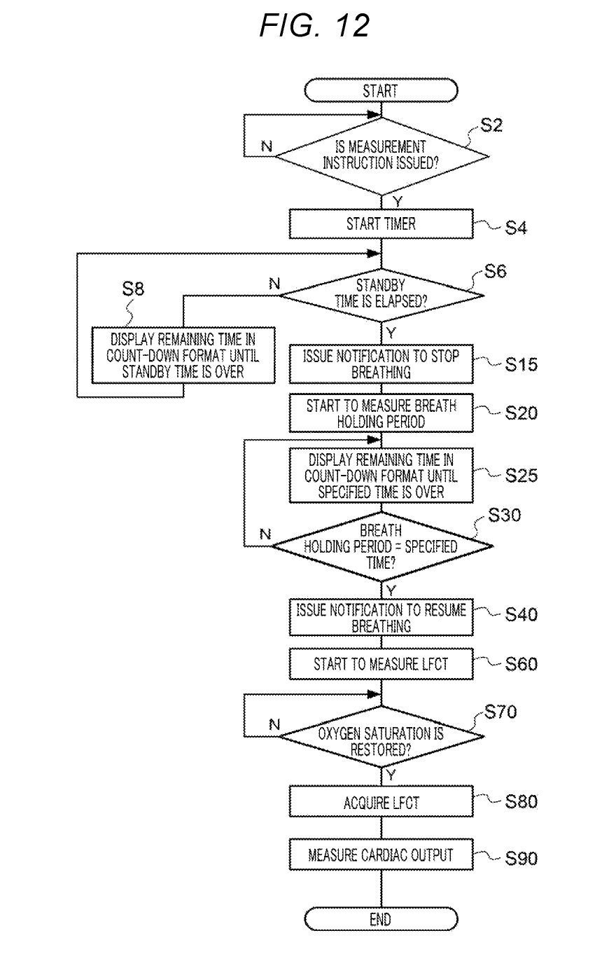

The operation of the biometric information measuring apparatus 10C or 10D will be described with reference to FIG. 12.

FIG. 12 is a flow chart showing an example of the biometric information measurement processing executed by the CPU 21 when the biometric information measuring apparatus 10C or 10D is activated.

The photoelectric sensor 11 is attached to a fingertip of the subject. Upon receiving the cardiac output measurement instruction, the biometric information measuring apparatus 10C continues to measure the oxygen saturation of the subject at least until the cardiac output measurement is completed. In the biometric information measuring apparatus 10C, the standby time is set in advance, for example, to n seconds.

The biometric information measurement programs specifying the biometric information measurement processing are stored in advance in the ROM 22 of the biometric information measuring apparatus 10C or 10D, for example. The CPU 21 of the biometric information measuring apparatus 10C or 10D reads the biometric information measurement program stored in the ROM 22, and executes the biometric information measurement processing.

The flow chart of the biometric information measurement processing shown in FIG. 12 differs from the flow chart shown in FIG. 10 in that steps S2 to S8, S15, and S25 are provided instead of step S10 for detecting the stoppage of breathing of the subject and step S50 for detecting the resumption of breathing of the subject.

First, in step S2, the CPU 21 determines whether or not the cardiac output measurement instruction is received from the user of the biometric information measuring apparatus 10C or 10D via the input unit 27.

When it is determined that the cardiac output measurement instruction is not received, the processing of step S2 is repeatedly executed to monitor the instruction from the user. On the other hand, when it is determined that the cardiac output measurement instruction is received, the processing proceeds to step S4.

In step S4, the CPU 21 starts the timer 15 to measure the standby time.

In step S6, the CPU 21 determines whether or not the timer value of the timer 15 reaches the standby time.

When the timer value of the timer 15 does not reach the standby time, the processing proceeds to step S8, and in step S8, the CPU 21 notifies the subject of the remaining time until the standby time is over. There is no limitation on the method of notifying the subject of the remaining time until the standby time is over, and for example, when the display unit 28 is connected to the biometric information measuring apparatus 10C or 10D, the remaining time until the standby time is over is displayed on the display unit 28. When the speaker unit is connected to the biometric information measuring apparatus 10C or 10D, the subject is notified of the remaining time until the standby time is over by voice. When the vibration unit is connected to the biometric information measuring apparatus 10C or 10D, the vibration unit is vibrated to notify the subject of the remaining time until the standby time is over through a sense of the subject.

Then, the processing proceeds to step S6, and the process of determining whether or not the timer value of the timer 15 reaches the standby time is repeatedly executed.

On the other hand, if it is determined in the determination processing of step S6 that the timer value of the timer 15 reaches the standby time, the processing proceeds to step S15.

When the time from receiving the cardiac output measurement instruction elapsed the standby time, the CPU 21 stops the timer 15 and notifies the subject of the stoppage of breathing in step S15. As a result, the CPU 21 assumes that the subject stops breathing, executes step S20 described above, and starts the timer 15 again to start measuring the breath holding period. That is, the biometric information measuring apparatus 10B starts measurement of the breath holding period without confirming the stoppage of breathing of the subject.

Then, in step S25, the CPU 21 notifies the subject of the remaining time until the specified time is over, using the notification method described in step S8, until it is determined in step S30 that the breath holding period reaches the specified time.

In addition, unlike the biometric information measuring apparatus 10A, the CPU 21 executes step S60 without confirming the resumption of breathing of the subject after the notification to resume breathing is issued in step S40, and subsequently starts the measurement of the LFCT.

Thereafter, steps S70 to S90 described above are executed, and the cardiac output of the subject is measured. Thus, the biometric information measurement processing shown in FIG. 12 ends.

In steps S8 and S25, the subject is notified of the remaining time until the standby time is over and the remaining time until the specified time is over, respectively, but the method of expressing the remaining time is not limited. For example, when the notification is performed before 10 seconds from the remaining time, the subject may be notified of the remaining time in a count-down format such as “10”→“9”→, . . . , “1”→“0”. Further, for example, when the notification is performed before 10 seconds from the remaining time, the subject may be notified of the remaining time in a count-up format such as “0”→“1”→, . . . , “9”→“10”.

Various modifications of the biometric information measurement processing in the first exemplary embodiment are also applied to the biometric information measuring apparatus 10C.

In the biometric information measurement processing shown in FIG. 12, the LFCT is measured from the difference between the time t₁ at which the resumption of breathing of the subject is detected and the time t₂ at which the inflection point of the oxygen saturation occurs. However, the CPU 21 may restart the timer 15 instead of storing the time t₁ in step S60 of FIG. 12, and may stop the timer 15 instead of storing the time t₂ at which the inflection point occurs in step S80. In this case, the cumulative time measured by the timer 15 from when the subject is notified of resumption of breathing to when the inflection point of the oxygen saturation occurs may be acquired as the LFCT.

In the biometric information measuring apparatus 10C or 10D, after the notification to resume breathing is issued, the measurement of the LFCT is started without confirming the resumption of breathing of the subject. Therefore, it is determined whether or not the period from when the notification to resume breathing is issued to when the subject actually resumes breathing is within the allowable delay period. When the period exceeds the allowable delay period, the modification for ending the biometric information measurement processing shown in FIG. 12 without measuring the LFCT is not applied to the apparatus 10C or 10D.

However, even if the notification to resume breathing is issued from the biometric information measuring apparatus 10C, the subject will resume breathing after recognizing that the notification to resume breathing is issued. Therefore, a delay time may occur between when the biometric information measuring apparatus 10C issues the notification to resume breathing and when the breathing is actually resumed.

Therefore, in the biometric information measuring apparatus 10C, the LFCT may be measured considering the delay time. Specifically, the CPU 21 may acquire the time obtained by subtracting the delay time from the LFCT acquired in step S80 of FIG. 12 as the final LFCT.

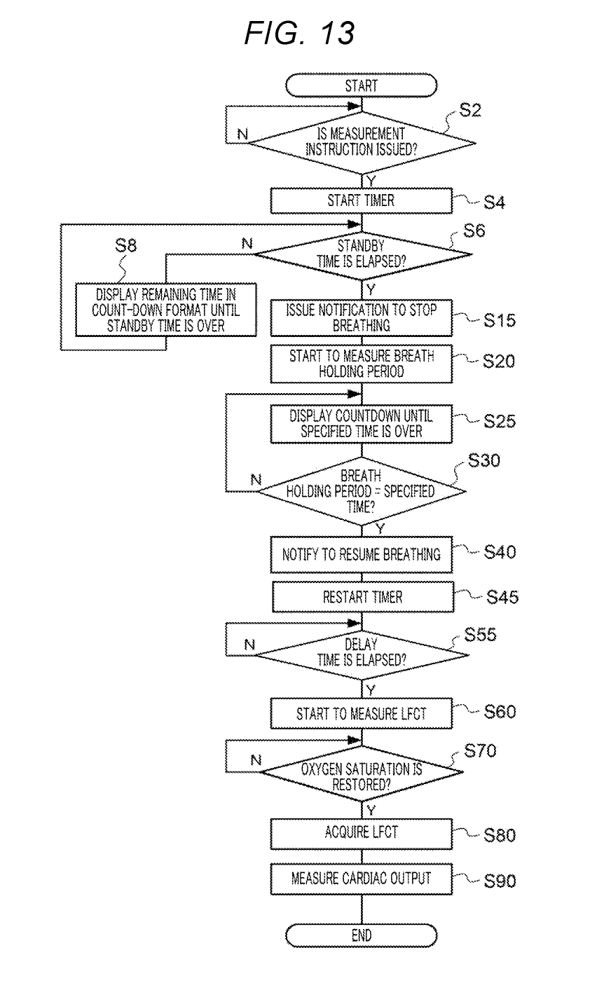

FIG. 13 is a flowchart showing an example of the biometric information measurement processing in consideration of the delay time accompanying the resumption of breathing.

The flowchart shown in FIG. 13 differs from the flowchart of the biometric information measurement processing shown in FIG. 12 in that steps S45 and S55 are added between steps S40 and S60.

After the notification to resume breathing is issued in step S40, the CPU 21 starts the timer 15 in step S45.

In step S55, the CPU 21 determines whether or not the timer value of the timer 15 started in step S40 reaches a predetermined delay time. The delay time represents a standard time from when the notification to resume breathing is issued to when the subject actually resumes breathing, and is the value obtained in advance by experiments using the actual biometric information measuring apparatus 10B for plural subjects.

If the timer value of the timer 15 does not reach the delay time in the determination processing of step S55, the processing of step S55 is repeatedly executed to monitor the timer value of the timer 15. On the other hand, when the timer value of the timer 15 reaches the delay time, the processing proceeds to step S60, and the LFCT is measured. That is, the biometric information measuring apparatus 10B that executes the biometric information measurement processing shown in FIG. 14 measures a time shorter by a delay time than the time from when the notification to resume breathing is issued to when the inflection point of the saturated oxygenation is detected as the LFCT.

Although the delay of the resumption of breathing has been described as an example here, the same can be applied to the stoppage of breathing. Therefore, steps S45 and S55 in FIG. 13 may be added between steps S15 and S20. The delay time, in this case, represents a standard time from when the notification to stop breathing is issued to when the subject actually stops breathing, and is the value obtained in advance by experiments using the actual biometric information measuring apparatus 10B for plural subjects.

As described above, the biometric information measuring apparatus 10D does not necessarily need to perform real-time processing for measuring the cardiac output of the subject during the measurement of the oxygen saturation.

For example, the CPU 21 stores the oxygen saturations of the respective times and the time information in the RAM 23 in association with each other at least in a predetermined period after the timer value of the timer 15 reaches the specified time, and temporarily ends the biometric information measurement processing shown in FIG. 12 without executing steps S70 to S90.

Then, when the CPU 21 receives a cardiac output measurement instruction from the user of the biometric information measuring apparatus 10D, the CPU 21 reads from the RAM 23 the oxygen saturation and the time information at the respective times after the timer value of the timer 15 reaches the specified time. Thereafter, the CPU 21 executes the processing including steps S70 to S90 of FIG. 12, detects the inflection point of the oxygen saturation from the read change of the oxygen saturation, acquires the LFCT from the difference between the time at which the timer value of the timer 15 reaches the specified time, that is, the time t₁ at which the subject resumed breathing and the time t₂ at which the inflection point of the oxygen saturation occurs, and measures the cardiac output using the acquired LFCT.

In the biometric information measuring apparatus 10C according to the third exemplary embodiment, the measurement of the breath holding period and the measurement of the LFCT are started without confirming the breathing state of the subject, and the cardiac output of the subject is measured. The biometric information measuring apparatus 10D receives the notifications of the stoppage of breathing and resumption of breathing in accordance with the timer value of the timer 15. When the apparatus 10D receives a notification of the resumption of breathing, the apparatus 10D measures the time from receiving the instruction until the inflection point of the oxygen saturation occurs and measures the oxygen circulation time and cardiac output. Therefore, the apparatus 10D also measures the oxygen circulation time and the cardiac output of the subject without using a detecting unit such as an airflow sensor for detecting the breathing state of the subject.

Although the present disclosure has been described using each embodiment, the present disclosure is not limited to the range described in each embodiment. Various changes or improvements can be added to the embodiments without departing from the spirit of the present disclosure, and the embodiments to which such changes or improvements are added are also included in the technical scope of the present disclosure. For example, the order of processing may be changed without departing from the spirit of the present disclosure.

Further, each of the embodiments shows an exemplary embodiment in which the biometric information measurement processing is realized by software as an example. Instead, processing equivalent to the flowcharts shown in FIG. 10, FIG. 12, and FIG. 13 may be implemented in, for example, an application specific integrated circuit (ASIC) and processed by hardware. In this case, the speed of the detection processing can be increased.

In the above-described embodiments, biometric information measurement programs are installed in the ROM 22. This is a non-limiting example. Alternatively, the biometric information measurement program may be provided in a form recorded on a non-transitory computer-readable storage medium. For example, the biometric information measurement program according to the present disclosure may be provided in a form in which the biometric information measurement program is recorded on an optical disc such as a Compact Disc (CD)-ROM or a Digital Versatile Disc (DVD)-ROM. The biometric information measurement program according to the present disclosure may be provided in a form in which the biometric information measurement program is recorded in a semiconductor memory such as a USB memory or a flash memory. Further, the biometric information measuring apparatuses 10A to 10D may acquire the biometric information measurement program according to the present disclosure from an external device connected to a communication line via the communication unit 29.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. A biometric information measuring apparatus comprising:
a notification unit that notifies a subject holding breath to resume breathing when a specified time to hold breath is reached; and
a measuring unit that measures an oxygen circulation time representing a time required for oxygen taken into a body of the subject by resumption of breathing by the subject to reach a predetermined site.

2. The biometric information measuring apparatus according to claim 1, further comprising:
a detecting unit that detects whether the subject is breathing, wherein
the notification unit issues the notification to resume breathing when an elapsed time from when the detecting unit detects a stoppage of breathing of the subject reaches the specified time.

3. The biometric information measuring apparatus according to claim 2, wherein
the measuring unit measures the oxygen circulation time when the breathing of the subject is detected by the detecting unit after the notification unit issues the notification to resume breathing.

4. The biometric information measuring apparatus according to claim 2, wherein
the detecting unit detects whether the subject is breathing by detecting an airflow in a mouth or nose of the subject.

5. The biometric information measuring apparatus according to claim 2, wherein
the detecting unit detects whether the subject is breathing by detecting a change in an amplitude of a pulse wave of the subject.

6. The biometric information measuring apparatus according to claim 2, wherein
the detecting unit detects whether the subject is breathing by detecting a movement of a chest of the subject.

7. The biometric information measuring apparatus according to claim 1, further comprising:
a receiving unit that receives information that indicates that breathing of the subject is stopped and is notified by an operation by the subject or a measurer who measures biometric information on the subject, wherein
the notification unit issues the notification to resume breathing when an elapsed time from when the receiving unit receives the information indicating that breathing of the subject is stopped reaches the specified time.

8. The biometric information measuring apparatus according to claim 7, wherein
the measuring unit measures the oxygen circulation time when the receiving unit receives information indicating that the subject starts breathing after the notification to resume breathing is issued.

9. The biometric information measuring apparatus according to claim 1, wherein
the measuring unit measures the oxygen circulation time when a period from when the notification unit issues the notification to resume breathing to when the subject resumes breathing is within a predetermined period.

10. The biometric information measuring apparatus according to claim 1, wherein
the measuring unit stops measuring the oxygen circulation time when a period from when the notification unit issues the notification to resume breathing to when the subject resumes breathing exceeds a predetermined period.

11. The biometric information measuring apparatus according to claim 1, wherein
the notification unit notifies the subject to stop breathing and issues the notification to resume breathing when an elapsed time from the notification to stop breathing reaches the specified time.

12. The biometric information measuring apparatus according to claim 11, wherein
the measuring unit measures the oxygen circulation time after the notification unit issues the notification to resume breathing.

13. The biometric information measuring apparatus according to claim 12, wherein
the measuring unit continuously measures the oxygen circulation time after the notification unit issues the notification to resume breathing.

14. The biometric information measuring apparatus according to claim 12, wherein
the measuring unit measures the oxygen circulation time after elapse of a predetermined time after the notification unit has issued the notification to resume breathing.

15. The biometric information measuring apparatus according to claim 11, wherein
the notification unit notifies the subject of a remaining time until the notification to stop breathing is issued.

16. The biometric information measuring apparatus according to claim 1, wherein
the notification unit notifies the subject of a remaining time until the specified time is reached while the subject is holding breath.

17. The biometric information measuring apparatus according to claim 1, wherein
the specified time is adjusted for each subject.

18. The biometric information measuring apparatus according to claim 1, wherein
the measuring unit measures the oxygen circulation time using an oxygen saturation indicating an oxygen concentration of blood at a predetermined site of the subject.

19. The biometric information measuring apparatus according to claim 18, wherein
the measuring unit measures the oxygen circulation time using the oxygen saturation indicating the oxygen concentration of blood in a finger of the subject as the predetermined site of the subject.

20. The biometric information measuring apparatus according to claim 1, further comprising:
an input device that receives an operation by the subject or a measurer who measures biometric information on the subject; and
a display device that displays the notification to resume breathing.

21. A non-transitory computer readable medium storing a program causing a computer to execute a process for biometric information measurement, the process comprising:
notifying a subject holding breath to resume breathing when a specified time to hold breath is reached; and
measuring an oxygen circulation time representing a time required for oxygen taken into a body of the subject by resumption of breathing by the subject to reach a predetermined site.

22. A biometric information measuring apparatus, comprising:
a receiving unit that receives a measurement instruction that instructs to measure time and is issued when a time measured by a measurement device reaches a specified time or when an input device is operated, and receives the measurement instruction by the time of occurrence of an inflection point where an oxygen concentration of blood at a predetermined site of a subject changes from decreasing to increasing; and
a measuring unit that measures an oxygen circulation time from when the receiving unit receives the measurement instruction to when the inflection point occurs.

23. The biometric information measuring apparatus according to claim 22, wherein
the receiving unit receives the measurement instruction from the input device operated by the subject or a measurer who measures biometric information on the subject in accordance with resumption of breathing of the subject.

24. The biometric information measuring apparatus according to claim 22, wherein
the receiving unit receives the measurement instruction when the time measured by the measurement device reaches the specified time representing a breath holding period.

25. The biometric information measuring apparatus according to claim 23, wherein
the measuring unit starts the measurement of time when the receiving unit receives the measurement instruction, and measures the oxygen circulation time using a cumulative time from when the receiving unit receives the measurement instruction to when the inflection point occurs, when the inflection point of the oxygen concentration of blood in the subject occurs during the measurement of time.

26. The biometric information measuring apparatus according to claim 25, wherein
the measuring unit determines, as the oxygen circulation time, a time obtained by subtracting a predetermined time from the cumulative time.

27. The biometric information measuring apparatus according to claim 23, wherein
the measuring unit acquires a reception time at which the receiving unit receives the measurement instruction, and measures the oxygen circulation time using a difference between the acquired reception time of the measurement instruction and an occurrence time of the inflection point.

28. The biometric information measuring apparatus according to claim 27, wherein
the measuring unit detects the inflection point from changes in the oxygen concentration of blood in the subject during the measurement of time, and determines the oxygen circulation time using, as the occurrence time of the inflection point, a time at which the inflection point is detected.

29. The biometric information measuring apparatus according to claim 27, wherein
the measuring unit stores, in a storage device, a time course of oxygen concentration over a predetermined period, and determines the oxygen circulation time using, as the occurrence time of the inflection point, a time corresponding to the inflection point obtained from the time course of oxygen concentration stored in the storage device.

30. The biometric information measuring apparatus according to claim 27, wherein
the measuring unit determines, as the oxygen circulation time, a time obtained by subtracting a predetermined time from a difference between the reception time of the measurement instruction and the occurrence time of the inflection point.

31. The biometric information measuring apparatus according to claim 22, wherein
the measuring unit acquires the inflection point using an oxygen saturation indicating the oxygen concentration of blood in the subject, and measures the oxygen circulation time.

32. The biometric information measuring apparatus according to claim 31, wherein
the measuring unit acquires the inflection point using the oxygen saturation indicating the oxygen concentration of blood in a finger of the subject, and measures the oxygen circulation time.

33. The biometric information measuring apparatus according to claim 22, further comprising:
the input device that receives an operation by the subject or a measurer who measures biometric information on the subject; and
a display device that displays that the specified time is reached.

34. A non-transitory computer readable medium storing a program causing a computer to execute a process for biometric information measurement, the process comprising:
receiving a measurement instruction that instructs to measure time and is issued when a time measured by a measurement device reaches a specified time or when an input device is operated, and receiving the measurement instruction by the time of occurrence of an inflection point where an oxygen concentration of blood at a predetermined site of a subject changes from decreasing to increasing; and
measuring an oxygen circulation time from when the measurement instruction is received to when the inflection point occurs.
